(19) United States
(12) Plant Patent Application Publication
(10) Pub. No.: US 2026/0271810 P1
(43) Pub. Date: Sep. 10, 2026

(54) Hydrangea Plant Named 'HMOGP08-0'

(50) ;

(71) Applicant: Steve Jones, Santa Monica, CA (US)

(72) Inventor: Steve Jones, Santa Monica, CA (US)

(21) Appl. No.: 19/549,965

(22) Filed: Feb. 25, 2026

Related U.S. Application Data
(60) Provisional application No. 63/768,971, filed on Mar. 8, 2025.

(51) Int. Cl. A01H 6/48; A01H 5/02

(52) U.S. Cl. CPC A01H 6/48; A01H 5/02
USPC PLT/250

(57) ABSTRACT

A new and distinct cultivar of Hydrangea plant named ‘HMOGP08-0’ is disclosed, characterized by very large, soft pink male flowers and rich deep rose female flowers. Flowers initiate on first year growth in both short daylight hours and long daylight hours. Flowers also initiate on first year growth without going through cold periods. Plants have a short production cycle of The new cultivar is a Hydrangea, typically produced as an outdoor ornamental plant.

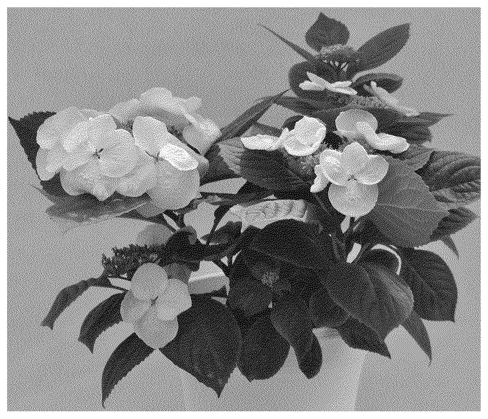

Latin name of the genus and species: Hydrangea spp.

Cultivar denomination: ‘HMOGP08-0’

### BACKGROUND OF THE INVENTION

The new cultivar is a product of a planned breeding program conducted by the inventor. Seeds germinated at a nursery in Somis, California for testing and selection. Cuttings were produced from selections in greenhouses. The crossing resulting in the new cultivar was made in Aug. 2021.

The seed parent is the cultivar Hydrangea ‘Deep Purple’, unpatented. The pollen parent is an unnamed, unpatented proprietary hybrid cultivar of Hydrangea.

The new cultivar was found and selected by the inventor in October 2023 at a nursery in Somis, California.

Asexual reproduction of the new cultivar ‘HMOGP08-0’ was first performed during October of 2023 in Somis, California, by terminal cuttings. Through subsequent propagation, multiple additional generations have been reproduced, having shown that the unique features of this cultivar are stable and reproduced true to type.

### SUMMARY OF THE INVENTION

The cultivar ‘HMOGP08-0’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘HMOGP08-0’. These characteristics in combination distinguish ‘HMOGP08-0’ as a new and distinct Hydrangea cultivar:
1. Flowers initiate on first year growth in both short daylight hours and long daylight hours.
2. Flowers initiate on first year growth without going through cold periods.
3. Male flower is very large, sepals 4 cm to 10 cm in length.
4. Distinctive soft pink male flowers and rich deep rose female flowers.
5. Fast flowering resulting in a shortened production cycle. Existing commercial comparisons require long days and winter cooling to initiate flowers. This means that even under ideal conditions of cooling, commercial varieties will take 6 weeks of cooling and an additional 8 to 12 weeks to initiate flowers. The cultivar described in this application will initiate flowers approximately 8 weeks after pinching under typical California conditions. Thus, it flowers quickly, when compared to existing commercial lines of Hydrangea.

### PARENT COMPARISONS

Plants of ‘HMOGP08-0’ are similar to plants of the seed parent in most horticultural characteristics, however, plants of ‘HMOGP08-0’ differ in the following:
1. Flowers of the new cultivar initiate on first year growth without long days, while flowers of the seed parent bloom mainly in late spring.
2. Flowers of the new cultivar initiate on first year growth without going through cold periods, while flowers of the seed parent perform better with a cooling period.
3. Male flowers of the new cultivar are very large, while the male flowers of the seed parent are ⅓ the size of the new selection.
4. Plants of the new cultivar flower sooner than plants of the seed parent.

Plants of ‘HMOGP08-0’ are similar to plants of the pollen parent in most horticultural characteristics, however, plants of ‘HMOGP08-0’ differ in the following:
1. Flowers of the new cultivar initiate on first year growth without long days, while flowers of the pollen parent bloom mainly in late spring.
2. Flowers of the new cultivar initiate on first year growth without going through cold periods, while flowers of the pollen parent perform better with a cooling period.
3. Male flowers of the new cultivar are very large, while the male flowers of the pollen parent are ⅓ the size of the new selection.
4. Plants of the new cultivar flower sooner than plants of the seed pollen parent.

### BRIEF DESCRIPTION OF THE PHOTOGRAPH

The accompanying photograph illustrates in full color a typical plant of ‘HMOGP08-0’ grown in Somis, California. This plant is approximately 1.5 years old, grown in a 2-gallon container. The photograph was taken using conventional techniques and although colors May appear different from actual colors due to light reflectance it is as accurate as possible by conventional photographic techniques.

### DETAILED BOTANICAL DESCRIPTION

In the following description, color references are made to the Royal Horticultural Society Colour Chart 2015, except where general terms of ordinary dictionary significance are used. The following observations and measurements describe ‘HMOGP08-0’ plants grown outdoors in Somis, California. The plants were about 2 to 3 years old. Temperatures ranged from 5° C. to 10° C. at night to 18° C. to 27° C. during the day. No artificial light, photoperiodic treatments were given to the plants. Measurements and numerical values represent averages of typical plant types.
Botanical classification: Hydrangea spp. ‘HMOGP08-0’
Propagation:
   Typical method.—Terminal cuttings.
   Root initiation.—5 to 7 days in the summer.
   Time to produce rooted cutting.—28 days in the summer.
   Roots.—Thin to medium, freely branching, fibrous, moderately dense. Beige to tan in color, not accurately measured with RHS chart.
Plant:
   Plant type.—Flowering deciduous perennial shrub.
   Age of plant described.—Approximately 7 months from a rooted cutting.
   Growth habit.—Rounded and upright.
   Height.—40 cm.
   Plant spread.—50 cm.
   Plant vigor.—Moderate.
   Stem.—Branching habit: Strong branching from a pinch. Branches occurring at mostly obtuse angles. Number of Lateral Branches: Average range 9 to 14. Shape: Round. Color: Immature: RHS Yellow-Green 144B. Mature: RHS Greyed-Purple 183A, streaked Greyed-Orange 165C and 165D. Length: Average 35 cm. Width: 7 to 12 mm. Strength: Strong. Aspect: Straight, occurring at an average 45° from center. Pubescence: None. Texture: Glabrous. Lenticels present.
   Lenticels.—Density: Sparse over majority of stem. Newest growth moderately dense. Color: RHS Greyed-Purple 187A. Size: 2 mm long 1 mm wide. Shape: Oval.
   Internode Length.—3 to 4.5 cm.
Foliage:
   Leaf.—Arrangement: Opposite. Shape: Ovate. Length: 14 to 16 cm. Width: 8.5 to 10.5 cm. Apex: Acuminate. Base: Obtuse. Margin: Dentate. Texture of top surface: Glabrous. Texture of bottom surface: Glabrous. Pubescence: None. Color: Young Foliage Upper Side: RHS Green 139A. Young Foliage Under Side: RHS Green 138A. Mature Foliage Upper Side: RHS Green 137A. Mature Foliage, Under Side: RHS Green 137C. 24.
   Venation.—Type: Pinnate. Color: Mature, Upper Side: RHS Yellow-Green N144B. Mature, Under Side: RHS Yellow-Green N144B.
   Petiole.—Length: 2.5 to 3 cm. Width: 4 mm. Color: Upper Side: RHS Green 143C. Under Side: RHS Green 143C. Texture: Upper Side: Glabrous. Under Side: Glabrous.
Flower:
   Natural flowering season.—Day length neutral, flowering begins approximately 13 to 16 weeks after planting during any time of the year without chill hours.
   Inflorescence.—Type: Lacecap. Compound umbel. Inflorescences per plant: Average 10.Quantity of flowers per inflorescence: Fertile flowers: 100-180. Sterile flowers: Average 3 to 5. Shape: Flattened disc. Height: 10 cm. Diameter: 12 to 18 cm. Fragrance: None detected.
   Sterile flower.—Bud shape: Round. Bud length: 29 mm. Bud diameter: 18 mm. Bud color: Near RHS Red 56D, flushed 55C. Aspect: Upright. Aging flowers very slightly pendulous. Flower depth: 25 mm. Flower Diameter: 7 to 11 cm. Persistence: Self-cleaning.
   Sterile flower petals.—Petals do not develop/open, or not present.
   Sterile flower sepal.—Arrangement: Cruciform, strongly overlapping. Number: 4. Shape: Wide deltate to irregular orbicular. Tip: Broad acute to obtuse. Base: Obtuse. Margin: Entire. Length: 4 to 10 cm. Width: 5 to 6 cm. Texture, Upper: Smooth. Texture, Lower: Smooth. Color: Upper surface at first opening: Near RHS Red 56D, flushed 55C near margins. Under surface at first opening: Near RHS Red 56D, flushed 55C near margins. Upper surface at maturity: Near RHS Red 56B, flushed Red-Purple 63D throughout, 63C near margins. Under surface at maturity: Near RHS Red 56D flushed Red-Purple 63D throughout, 63C near margins.
   Sterile flower pedicel.—Length: 3 to 4 cm. Diameter: 2.5 mm. Angle: About 90°, straight aspect. Strength: Flexible. Texture: Glabrous. Color: RHS Red-Purple 63C; upper section flushed 63B, lower section Yellow-Green 144D.
   Fertile flower.—Rotate flower composed of petals and noticeably protruding stamens. Petals do not unfurl on all flowers. Approximately 70% of flowers remain with furled petals and protruding stamens. Bud shape: Sphere. Bud length: 5 mm. Bud diameter: 5 mm. Bud color: RHS Yellow-Green 144D, flushed Purple-Pink 55A and 55B. Flower aspect: Upright. Flower shape: Rotate. Flower diameter: 8 mm, petals only when fully unfurled; including protruding stamens 14 mm. Flower length: 7 mm petals only; including protruding stamens 10 mm. Persistence: Self-cleaning.
   Fertile flower petals.—Arrangement: Rotate. Quantity: 5. Length of petal: 3 mm to 6 mm. Width of petal: 2 mm to 3 mm. Apex: Acute. Shape of petal: Elliptic. Petal margin: Entire. Petal base: Truncate. Petal Texture: Smooth, upper and under side. Color: Upper surface at first opening: Base RHS Yellow-Green 144D, upper ⅔ Red-Purple N57C. Under surface at first opening: Base RHS Yellow-Green 144D, upper ⅔ Red-Purple N57D. Upper surface at maturity: RHS Red-Purple 72B. Under surface at maturity: RHS Red-Purple 72C.
   Fertile flower sepal.—Minute and inconspicuous, about 1 mm long and 2 mm wide, colored near RHS Red 56-Purple 65A, upper and lower surfaces.
   Fertile flower pedicel.—Angle: About 35°. Strength: Flexible. Length: Average 5 mm. Width: 2 mm. Texture: Glabrous. Color: Near RHS Red 55D.
Reproductive organs:
   Sterile flowers.—Androecium commonly present. Somewhat mis-formed gynoecium irregularly present.
   Stamens.—Number: 4 to 5. Filament length: 5 mm. Filament color: RHS Greyed-Purple N186A.
   Anthers.—Shape: Globular. Length: 1 mm. Color: RHS Greyed-Purple N187D. Pollen: Not observed.
   Pistil.—Number: 1. Length: 2 to 4 mm. Style: Length: 2 to 3 mm. Color: Near RHS Red 55C. Ovary: About 0.5 mm in diameter and length. Colored near RHS Red 55D. Stigma: Shape: Bi-lobed. Color: RHS Greyed-Purple N186A.
   Fertile flowers.—.
   Stamens.—Number: About 5. Filament length: 8 mm to 12 mm. Filament color: RHS Red-Purple 71A.
   Anthers.—Shape: Globular. Length: 1 mm. Color: RHS Red-Purple 71D. Pollen color: RHS White N155A. Pollen amount: Moderate.
   Pistil.—Number: 3. Length: 3 mm to 4 mm. Style: Length: 2 mm to 3 mm. Color: Near RHS Red-Purple N74D. Ovary color: Near RHS Red 55D. Stigma: Shape: Bi-lobed. Color: Near RHS Red 55C.
Other characteristics:
   Seeds and fruits.—Not observed to date.
   Disease/pest resistance.—Neither resistance nor susceptibility to normal diseases and pests of Hydrangea have been observed.
   Temperature tolerance.—Low temperature tolerant to USDA Zones 5.

What is claimed is:

1. A new and distinct cultivar of Hydrangea plant named ‘HMOGP08-0’ as herein illustrated and described.
